(19) United States
(12) Patent Application Publication
(10) Pub. No.: US 2016/0000399 A1
(43) Pub. Date: Jan. 7, 2016

(54) METHOD AND APPARATUS FOR ULTRASOUND NEEDLE GUIDANCE

(71) Applicant: General Electric Company, Schenectady, NY (US)

(72) Inventors: Menachem Halmann, Milwaukee, WI (US); Eunji Kang, Brookfield, WI (US)

(21) Appl. No.: 14/322,115

(22) Filed: Jul. 2, 2014

(51) Int. Cl. A61B 8/08; A61B 19/00

(52) U.S. Cl. CPC A61B 8/0841; A61B 19/5225; A61B 2019/4847; A61B 2560/0276

(57) ABSTRACT

A method and apparatus of ultrasound guidance for interventional procedures involving a needle includes acquiring ultrasound data from a region of interest, positioning the needle with respect to the region of interest, displaying an image based on the ultrasound data, calculating a risk of bending for the needle, and presenting the risk of bending for the needle.

### FIELD OF THE INVENTION

This disclosure relates generally to a method and apparatus for providing ultrasound guidance for interventional procedures involving a needle.

### BACKGROUND OF THE INVENTION

Ultrasound imaging is used to acquire images of tissue in order to identify an anatomical target. Additionally, ultrasound imaging is used to help predict and guide the placement of a needle during interventional procedures. For example, ultrasound guidance is often used to guide procedures such as positioning a biopsy needle, administering a nerve block, or placing a peripherally inserted central catheter (PICC) line. During an interventional procedure involving a needle, a clinician is concerned about the location and future trajectory of the needle that will be inserted into the patient. The clinician needs to clearly understand the needle position and trajectory for both patient safety and clinical effectiveness. In order to complete a successful interventional procedure, the clinician must accurately position the needle tip in the desired anatomy while avoiding causing any undue tissue damage during the process of inserting and positioning the needle. In addition to avoiding particular anatomical regions, oftentimes it is desirable to position the needle in extremely close proximity to other structures. In order to safely accomplish an interventional ultrasound procedure, the clinician needs to position the needle to obtain a desired insertion trajectory prior to insertion of the needle.

Conventional techniques for ultrasound needle guidance involve tracking the position of the needle through the use of a tracking system, such as an electromagnetic or an optical tracking system. A sensor is typically attached to either a tip of the needle or to a hub of the needle, and then a processor calculates the position of the needle based on data from the sensor. Conventional techniques are able to generate a predicted path for the needle based on the position data and display this predicted path on the ultrasound image.

For reasons of patient comfort and safety, it is generally desired to use as thin of a needle as possible when performing an interventional needle procedure. However, when using a thin needle with a small diameter (i.e. a higher gauge), there exists a significant risk that the needle will bend and, as a result, the path will deviate significantly from the predicted path. Depending upon the anatomy surrounding the predicted path, it may be extremely important for the clinician to be aware of situations with significant risk of bending the needle prior to insertion of the needle.

For these and other reasons an improved method and apparatus for ultrasound guidance for interventional procedures involving a needle is desired.

### BRIEF DESCRIPTION OF THE INVENTION

The above-mentioned shortcomings, disadvantages and problems are addressed herein which will be understood by reading and understanding the following specification.

In an embodiment, a method of ultrasound guidance for interventional procedures involving a needle includes acquiring ultrasound data from a region of interest, positioning the needle with respect to the region of interest, displaying an image based on the ultrasound data, calculating a risk of bending for the needle, and presenting the risk of bending for the needle.

In another embodiment, a method of ultrasound guidance for interventional procedures involving a needle includes acquiring ultrasound data from a region of interest, positioning the needle with respect to the region of interest, and acquiring position data during the process of positioning the needle. The method includes calculating a risk of bending for the needle based on the position data during the process of positioning the needle, and displaying an image based on the ultrasound data. The method includes displaying a graphic on the image representing the risk of bending. The method includes modifying the graphic in response to an increase or a decrease in the risk of bending during the process of positioning the needle.

In another embodiment, an apparatus for providing ultrasound guidance for interventional procedures involving an needle includes a needle tracking system that provides needle position data. The apparatus includes an ultrasound imaging system including a processor, a probe, and a display device. The processor is configured to receive needle position data from the needle tracking system and control the ultrasound imaging system to acquire ultrasound data from a region of interest with the probe. The processor is configured to generate an image based on the ultrasound data, display the image on the display device, calculate a risk of bending for the needle, and display a graphic on the image representing the risk of bending.

Various other features, objects, and advantages of the invention will be made apparent to those skilled in the art from the accompanying drawings and detailed description thereof.

### BRIEF DESCRIPTION OF THE DRAWINGS

FIG. 1 is a schematic representation of an apparatus for providing ultrasound guidance for interventional procedures in accordance with an embodiment;

FIG. 2 is schematic representation of a needle in accordance with an embodiment;

FIG. 3 is schematic representation of a probe in accordance with an embodiment;

FIG. 4 is a flow chart in accordance with an embodiment;

FIG. 5 is a schematic representation of a coordinate system used to track a needle in accordance with an embodiment;

FIG. 6 is a flow chart in accordance with an embodiment;

FIG. 7 is a schematic representation of a screenshot in accordance with an embodiment;

FIG. 8 is a schematic representation of a screenshot in accordance with an embodiment;

FIG. 9 is a schematic representation of a screenshot in accordance with an embodiment;

FIG. 10 is a schematic representation of a screenshot in accordance with an embodiment;

FIG. 11 is a schematic representation of a screenshot in accordance with an embodiment;

FIG. 12 is a schematic representation of a screenshot in accordance with an embodiment; and

FIG. 13 is a schematic representation of a screenshot in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

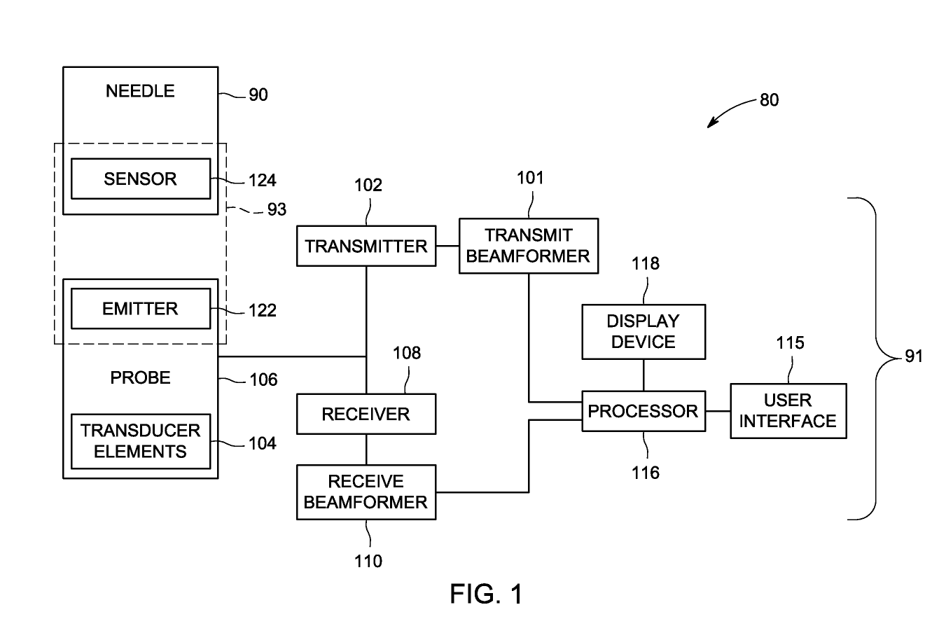

FIG. 1 is a schematic diagram of an apparatus 80 in accordance with an embodiment. FIG. 1 also includes a needle 90. The apparatus 80 includes an ultrasound imaging system 91 and a needle tracking system 93. The needle tracking system 93 includes an emitter 122 and a sensor 124. The emitter 122 is configured to emit some type of energy and the sensor 124 is configured to detect the energy from the emitter 122. For example, the emitter 122 may be an electromagnetic filed generator or a magnetic sensor board and the sensor 124 may comprises one or more coils adapted to detect the strength and orientation of the magnetic field. The needle tracking system 93 will be discussed in additional detail hereinafter. The ultrasound imaging system 91 includes a transmit beamformer 101 and a transmitter 102 that drive transducer elements 104 within a probe 106 to emit pulsed ultrasonic signals. A variety of geometries of probes 106 and transducer elements 104 may be used. The pulsed ultrasonic signals are back-scattered from structures such as blood cells or muscular tissue to produce echoes that return to the transducer elements 104. The echoes are converted into electrical signals, or ultrasound data, by the transducer elements 104 in the probe 106 and the electrical signals are received by a receiver 108 and then beamformed by the receive beamformer 110. The ultrasound data may comprise 2D ultrasound data or 3D ultrasound data. According to other embodiments, the probe 106 may contain electronic circuitry to do all or part of the transmit beamforming and/or the receive beamforming. For example, all or part of the transmit beamformer 101, the transmitter 102, the receiver 108 and the receive beamformer 110 may be disposed within the probe 106 according to other embodiments. The terms “scan” or “scanning” may also be used in this disclosure to refer to acquiring ultrasound data through the process of transmitting and receiving ultrasonic signals. For purposes of this disclosure, the term “ultrasound data” may include data that was acquired or processed by an ultrasound system. Additionally, the term “data” may also be used in this disclosure to refer to either one or more datasets. The electrical signals representing the received echoes are passed through the receive beamformer 110 that outputs ultrasound data. The receive beamformer 110 may be either a conventional hardware beamformer or a software beamformer according to various embodiments. If the receive beamformer 110 is a software beamformer, it may comprise one or more of the following components: a graphics processing unit (GPU), a microprocessor, a central processing unit (CPU), a digital signal processor (DSP), or any other type of processor capable of performing logical operations. The receive beamformer 110 may be configured to perform conventional beamforming techniques as well as techniques such as retrospective transmit beamforming (RTB). A user interface 115 may be used to control operation of the ultrasound imaging system 91. The user interface 115 may include one or more controls such as a keyboard, a rotary, a mouse, a trackball, a track pad, and a touch screen. The user interface 115 may, for example, be used to control the input of patient data, to change a scanning parameter, or to change a display parameter.

The ultrasound imaging system 91 also includes a processor 116 in electronic communication with the probe 106, the display device 118, the transmitter 102, the transmit beamformer 101, and the receive beamformer. The processor 116 may control the transmit beamformer 101, the transmitter 102 and, therefore, the ultrasound beams emitted by the transducer elements 104 in the probe 106. The processor 116 may also process the ultrasound data into images for display on a display device 118. According to an embodiment, the processor 116 may also include a complex demodulator (not shown) that demodulates the RF ultrasound data and generates raw ultrasound data. The processor 116 may be adapted to perform one or more processing operations on the ultrasound data according to a plurality of selectable ultrasound modalities. The ultrasound data may be processed in real-time during a scanning session as the echo signals are received. For the purposes of this disclosure, the term “real-time” is defined to include a process that is performed without any intentional delay, such as process that is performed with less than a 500 mS delay. Additionally or alternatively, the ultrasound data may be stored temporarily in a buffer (not shown) during a scanning session and processed in less than real-time in a live or off-line operation. Some embodiments may include multiple processors (not shown) to handle the processing tasks. For example, a first processor may be utilized to demodulate and decimate the RF signal while a second processor may be used to further process the data prior to displaying an image. It should be appreciated that other embodiments may use a different arrangement of processors to handle the processing tasks. For embodiments where the receive beamformer 110 is a software beamformer, the processing functions attributed to the processor 116 and the software beamformer hereinabove may be performed by a single processor such as the receive beamformer 110 or the processor 116. Or, the processing functions attributed to the processor 116 and the software beamformer may be allocated in a different manner between any number of separate processing components.

The ultrasound imaging system 91 may continuously acquire ultrasound data at a frame rate of, for example, 10 Hz to 30 Hz. Images generated from the ultrasound data may be refreshed at a similar frame rate. Other embodiments may acquire and display ultrasound data at different rates. For example, some embodiments may acquire ultrasound data at a frame rate of less than 10 Hz or greater than 30 Hz depending on the parameters used for the data acquisition. A memory (not shown) may be included for storing processed frames of acquired ultrasound data. The memory should be of sufficient capacity to store at least several seconds of ultrasound data. The memory may include any known data storage medium.

Optionally, embodiments of the present invention may be implemented utilizing contrast agents. Contrast imaging generates enhanced images of anatomical structures and blood flow in a body when using ultrasound contrast agents such as microbubbles. After acquiring ultrasound data while using a contrast agent, the processor 116 may separate harmonic and linear components, enhance the harmonic component, and generate an ultrasound image by utilizing the enhanced harmonic component. Separation of harmonic components from the received signals is performed using suitable filters. The use of contrast agents for ultrasound imaging is well-known by those skilled in the art and will therefore not be described in further detail.

In various embodiments of the present invention, ultrasound data may be processed by different mode-related modules (e.g., B-mode, Color Doppler, M-mode, Color M-mode, spectral Doppler, TVI, strain, strain rate, and the like) to form 2D or 3D image frames. The frames are stored and timing information indicating the time when the data was acquired in memory may be recorded. The modules may include, for example, a scan conversion module to perform scan conversion operations to convert the image frames from coordinate beam space to display space coordinates. A video processor module may be provided that reads the image frames from a memory and displays the image frames in real-time while a procedure is being carried out on a patient. A video processor module may store the image frames in an image memory, from which the images are read and displayed.

The needle tracking system 93 is schematically represented in FIG. 1. Components of the needle tracking system 93 may be integrated into the ultrasound imaging system 91, as shown in FIG. 1, or the needle tracking system 93 may comprise components that are separate from the ultrasound imaging system 91. According to the embodiment shown in FIG. 1, the needle tracking system 93 is a magnetic tracking system and it includes an emitter 122 disposed in the probe 106 and a sensor 124 disposed in the needle 90. According to an exemplary embodiment, the emitter 122 may comprise a magnetic sensor board. The magnetic sensor board includes a magnetic field generator configured to emit an electromagnetic field of a known direction and intensity. The sensor 124 disposed in the needle 90 may include three sets of coils, where each set of coils is disposed orthogonally to the two other sets of coils. For example, a first set of coils may be disposed along an x-axis, a second set may be disposed along a y-axis, and a third set may be disposed along a z-axis. Different currents are induced in each of the three orthogonal coils by the electromagnetic field generated from the magnetic field generator 96. By detecting the currents induced in each of the coils, position and orientation information may be determined from the sensor 124. According to an embodiment, the processor 116 is in electronic communication with the needle tracking system 93. For example, the probe 106 may be connected to the processor 116 via either a wired or a wireless connection. Likewise, position data from the sensor 124 may be communicated to the processor 116 via either a wired connection or through wireless techniques. The processor 116 is able to determine the position and orientation of the probe 106 based on the data from the sensor 124. In other embodiments, the emitter 122 may be located somewhere other than the probe 106. For example, the needle tracking system may use a stationary field emitter and both the probe 106 and the needle 90 may include sensors configured to detect the strength and orientation of the magnetic field. Additionally, it is conceivable that the needle 90 may house the transmitter 102 and that the receiver may be disposed in the probe 106. However, according to the exemplary embodiment, the sensor 124 is disposed in the needle 90 for ease of packaging considering the smaller form factor of the needle 90. Other embodiments may use different types of tracking systems. For example, an optical tracking system using light emitting diodes (LEDs) or reflectors and a camera system may be used to determine the relative position of the needle 90. Magnetic and optical tracking systems are well-known by those skilled in the art and, therefore, will not be described in additional detail.

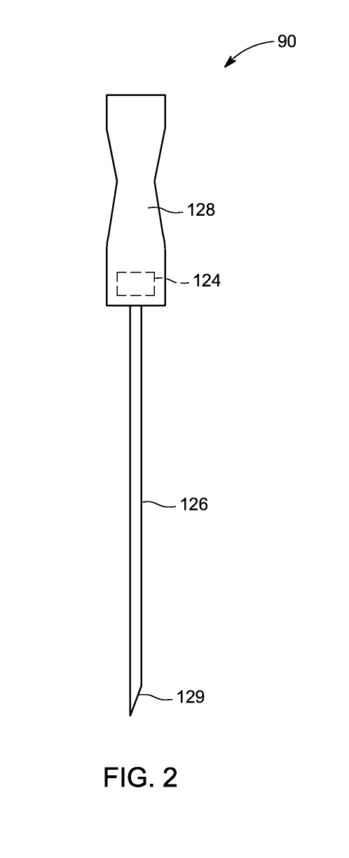

FIG. 2 is a schematic representation of the needle 90 shown in FIG. 1. The needle 90 includes a hollow tube 126, a hub 128, and the sensor 124. The hub 128 is configured to be grasped and manipulated by a clinician or user. According to an exemplary workflow, all positional adjustments of the needle 90, including inserting and withdrawing the needle 90, are the result of movements applied through the hub 128. As described previously with respect to FIG. 1, the sensor 124 may comprise a electromagnetic sensor according to an embodiment. The needle 90 also includes a needle tip 129.

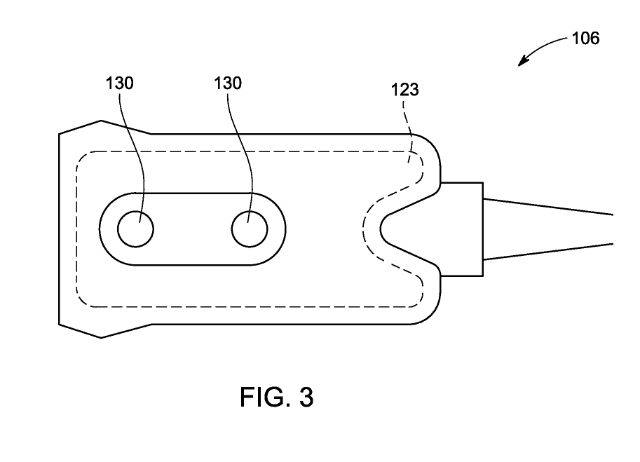

FIG. 3 is a schematic representation of the probe 106 in accordance with an exemplary embodiment. The probe 106 shown in FIG. 3 is a linear array probe, although it should be appreciated that any type or configuration of probe may be used with the ultrasound imaging system 91. The probe 106 is an exemplary embodiment where the emitter comprises a sensor board 123. The sensor board 123 is depicted in a dashed line because it is positioned internally within the probe 106. The probe 106 includes buttons 130 to control common imaging commands such as freeze, start, stop, or gain.

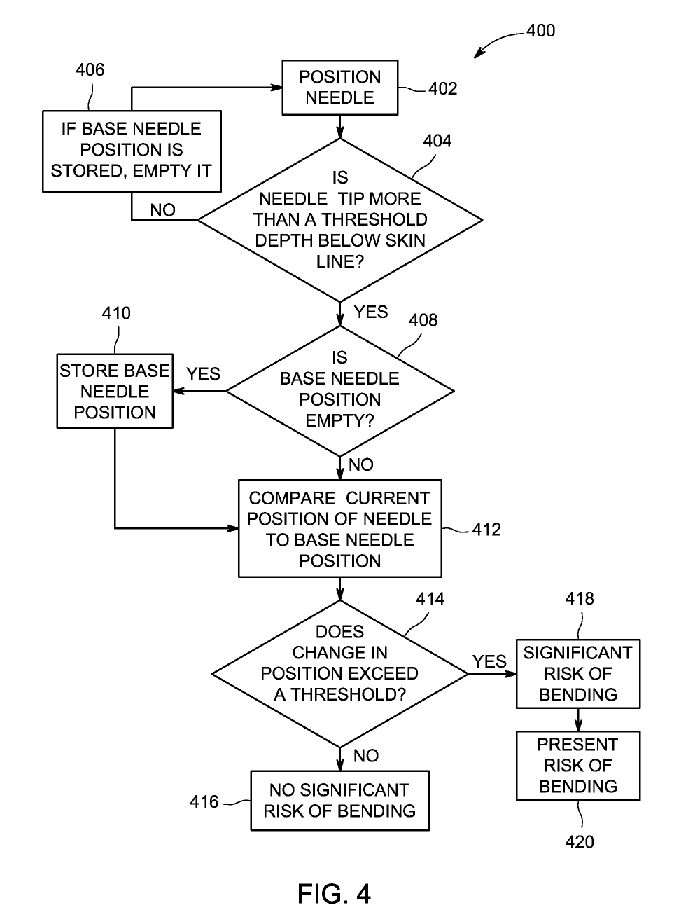

FIG. 4 is a flow chart of a method 400 in accordance with an embodiment. The individual blocks represent steps that may be performed in accordance with the method 400. Additional embodiments may perform the steps shown in a different sequence and/or additional embodiments may include additional steps not shown in FIG. 4. The technical effect of the method 400 is the calculation and presentation of the risk of bending for a needle.

The method 400 will be described according to an exemplary embodiment where the method 400 is implemented with the apparatus 80 shown in FIG. 1. According to an exemplary embodiment, the method 400 may be performed while the ultrasound imaging system 91 is in the process of acquiring ultrasound data from a region of interest and displaying one or more images based on the ultrasound data. The region of interest may include, for example, target tissue for the needle 90. The ultrasound data would most commonly comprise b-mode data, but the ultrasound data may comprise any other mode of data according to various embodiments.

At step 402, a clinician positions the needle 90 while the probe 106 is held stationary with respect to a patient (not shown). The clinician may position the needle 90 with respect to a region-of interest. The processor 116 may receive position data for the needle 90 either continuously or at regular intervals during the method 400. For example, the sensor 124 may push position data to the processor 116 at regularly defined intervals, such as every 50-100 mS. It should be appreciated that the position data may be updated at different intervals according to other embodiments.

At step 404, the processor 116 determines, based on the most recently acquired position data from the needle 90, if the needle tip 129 has been inserted to a depth deeper than a threshold depth below the patient's skin line. The threshold depth may be from 1-3 cm according to an exemplary embodiment, but other threshold depths may be used according to other embodiments. If the needle tip 129 has not exceeded the threshold depth, the method advances to step 406. At step 406, the processor 116 performs a conditional operation; if a base needle position has been stored in a memory or buffer, the processor 116 empties the base needle position. The base needle position represents a reference needle position with respect to an intended trajectory. The base needle position will be described in additional detail hereinafter.

If, at step 404, the needle tip has exceeded the threshold depth, the method 400 advances to step 408. At step 408, the processor 116 determines if the base needle position is empty (does not contain a value) or full (contains a value). If the base needle position is empty, the method 400 advances to step 410, where the most recent needle position is stored as the base needle position. After step 410, the method 400 advances to step 412. Or, if the base needle position is full at step 408, the method 400 advances to step 412. As step 412, the processor 116 compares the current position of the needle 90 to the base needle position that was stored at a previous step.

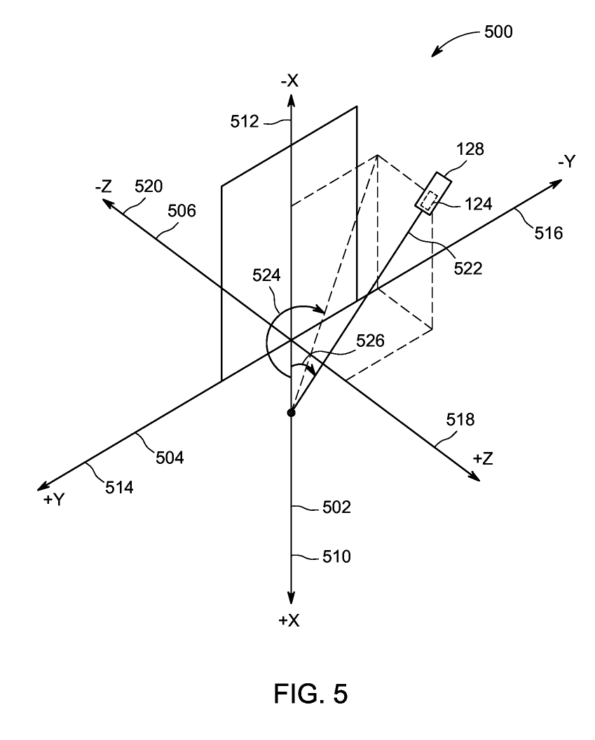

FIG. 5 is a schematic representation of a coordinate system 500 according to an exemplary embodiment. Position data for the needle 90 may be calculated with respect to the coordinate system 500. The coordinate system includes an x-axis 502, a y-axis 504, and a z-axis 506. Furthermore, each axis is divided into a positive axis and a negative axis; the coordinate system 500 includes a +X axis 510, a −X axis 512, a +Y axis 514, a −Y axis 516, a +Z axis 518, and a −Z axis 520. A needle axis 522 is also shown on the coordinate system 500. According to an embodiment, an x-y plane is defined by the position of the sensor board 123 in the probe 106. The position data for the sensor 124 in the needle 90 is therefore defined with respect to the X-Y plane. The hub 128 including the sensor 124 is shown in the coordinate system 500. According to an exemplary embodiment, the position data may include a phi angle 524 and a theta angle 526. The phi angle 524 is defined to include the rotation angle measured between the +X axis and the projection of needle axis 522 onto the X-Y plane. The theta angle 526 is defined to include the angle between the X-Y plane and the needle axis 522. Is should be appreciated that this is merely an exemplary coordinate system, and that any other coordinate system may be used according to other embodiments.

Those skilled in the art should appreciate that the clinician may be manipulating the needle 90 while the method 400 is being performed. For example, the clinician may position the needle 90 in order to align a projected trajectory of the needle 90 with an intended trajectory or an intended target. Or, the clinician may be actively in the process of inserting the needle 90 into a patient. At step 414, the processor 116 determines if the change in position exceeds a threshold. For example, the processor 116 may compare the phi angle 524 and the theta angle 526 for the needle 90 in its current position with the phi angle 524 and theta angle 526 of the base needle position. If the change in the phi angle, hereinafter delta phi, or the change in theta angle, hereinafter delta theta, exceeds the threshold, then the processor proceeds to step 418. The processor 116 may also compare the combination of delta phi and delta theta in order to determine if the change in position for the probe 92 exceeds a threshold at step 414.

Referring to FIG. 5, it is desired that the clinician inserts the needle 90 in an axial direction (i.e. in a direction along the length of the needle) when inserting the needle 90 into the patient. Any movement of the hub 128 in a non-axial direction is undesired and increases the risk of bending for the needle 90. By analyzing delta phi and delta theta for the needle 90 in a current position compared to the base needle position at step 414, the processor 116 is able to calculate a risk of bending for the needle 90. If delta phi and delta theta are smaller than the threshold, it may be assumed that the current position of the needle 90 is still substantially aligned with the needle axis 522 as established in the base needle position. However, if delta phi, delta theta, or the combination of delta phi and delta theta exceed the threshold, it may be assumed that the hub 128 is currently positioned in a manner that is either actively causing the needle 90 to bend or that would be likely to cause the needle 90 to bend if the needle 90 were inserted along its current trajectory. The processor 116 may also use additional factors when calculating the risk of bending such as a gauge of the needle, a stiffness of the needle 90 (which may be related to the gauge of the needle 90), and whether or not the needle has penetrated the skin. Any movement of the needle 90 or the hub 128 of the needle 90 in a non-axial direction may increase the risk of bending. However, higher gauge needles, more flexible needles, and situations where the needle 90 has already penetrated the patient's skin can all lead to an increased risk of bending. The processor 116 may use some or all of these variables to more accurately calculate the risk of bending for specific situations.

Referring back to FIG. 4, if the change in position is less than the threshold at step 414, the processor 116 determines that there is minimal risk of bending for the needle 90. On the other hand, if the change in the position of the needle 90 exceeds the threshold, the method 400 advances to step 418, and the processor 116 determines that there is a significant risk of bending. At step 420, the processor 116 presents the risk of bending. The risk of bending may be presented in many different ways according to various embodiments. For example, the processor 116 may present the risk of bending with one or more of the following techniques: displaying a graphic on the image, displaying a text-based warning or message, and playing an audible warning. Displaying a graphic on the image may comprise displaying an icon. Various embodiments showing different ways to present the risk of bending will be described hereinafter. According to an exemplary embodiment, some or all of the steps in the method 400 may be iteratively repeated during an ultrasound guided interventional procedure. For example, steps 412, 414, and steps 416 or 418 and 420 may be repeated if the processor 116 is still relying on the original base needle position. According to other embodiments, the entire method 400 may be iteratively repeated. It should be understood that one or more steps of the method 400 may not be performed during every iteration since the method 400 includes a number of conditional steps. According to an embodiment, the method 400 may be iteratively performed at a present interval, or the method 400 may be repeated at different refresh rate depending upon the capabilities and current processing load being handled by the processor 116.

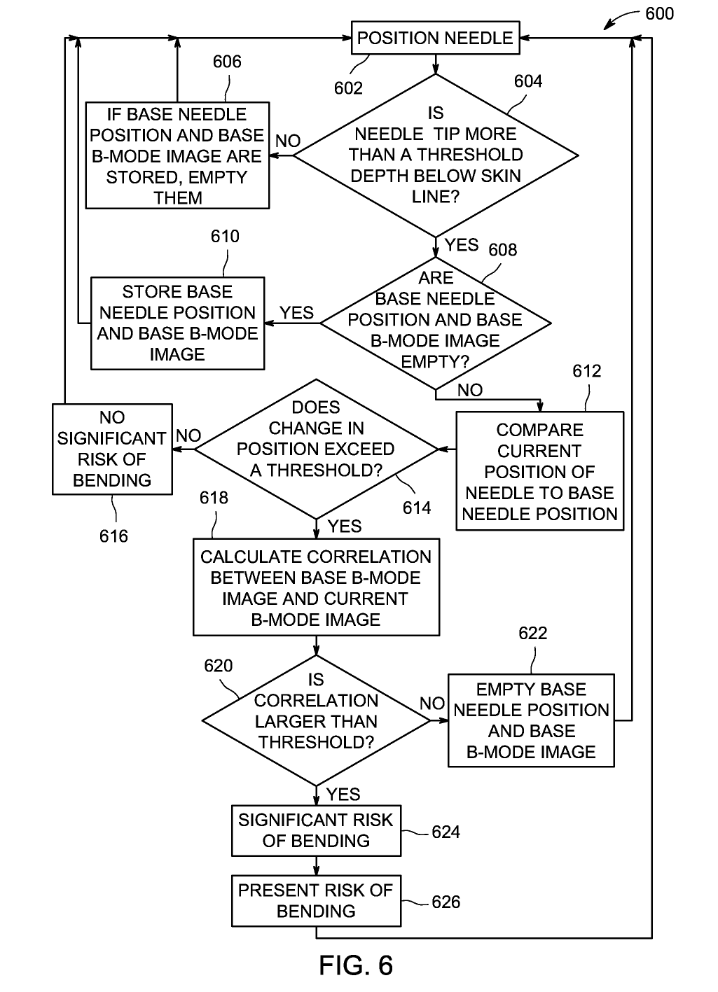

FIG. 6 is a flow chart of a method 600 in accordance with an embodiment. The individual blocks represent steps that may be performed in accordance with the method 600. Additional embodiments may perform the steps shown in a different sequence and/or additional embodiments may include additional steps not shown in FIG. 6. The technical effect of the method 600 is the calculation and presentation of the risk of bending for a needle. The method 600 will be described according to an exemplary embodiment where the method 600 is implemented with the apparatus 80 shown in FIG. 1. The method 600 may be performed while the ultrasound imaging system 91 is acquiring ultrasound data from a region of interest and displaying one or more images based on the ultrasound data. The region of interest may include a target tissue for the needle 90. The ultrasound data may comprise b-mode data or any other mode of ultrasound data.

At step 602, a clinician positions the needle 90 with respect to a patient (not shown). According to an exemplary embodiment, the processor 116 may receive position data for the needle 90 from the needle tracking system 93 either continuously or at regular intervals during the method 600. For example, the sensor 124 may push position data to the processor 116 at regularly defined intervals.

At step 604, the processor 116 determines, based on the most recently acquired position data from the needle 90, if the needle tip 129 has been inserted to a depth deeper than a threshold depth below the patient's skin line. The threshold depth may be from 1-3 cm according to an exemplary embodiment, but other threshold depths may be used according to other embodiments. If the needle tip 129 has not exceeded the threshold depth, the method advances to step 606. At step 606, the processor 116 performs a conditional operation; if a base needle position and a base b-mode image are stored in a memory or a buffer, the processor 116 empties the base needle position and the base b-mode image. The base needle position represents a reference needle position with respect to an intended trajectory. The base needle position will be described in additional detail hereinafter. The base b-mode image may comprise a static b-mode image.

If the needle tip 129 is deeper than the threshold beneath the skin line, then the method 600 advances to step 608. At step 608, the processor 116 determines if the base needle position and the base b-mode image are empty in the memory or buffer. If the base b-mode image and the base needle position are empty, the method 600 advances to step 610, where the processor 116 stores the base needle position and the base b-mode image in the memory or buffer. After step 610 has been performed, the method 600 advances to step 602. If the base needle position and the base b-mode image are not empty, the method 600 advances to step 612. At step 612, the processor 116 compares the current position of the needle 90 to the base needle position. Next, at step 614, the processor 116 determines if the change in position for the needle 90 exceeds a threshold. Step 614 is similar to the previously described step 414 of the method 400 and will not be described in additional detail with respect to the method 600. If the change in the needle position does not exceed the threshold, then the processor 116 determines that there is not significant risk of bending at step 616 and the method 600 advances to step 602.

Referring back to step 614, if the change in position of the needle 90 does exceed the threshold, then the method advances to step 618. At step 618, the processor 116 calculates the correlation between the base b-mode image and the current b-mode image. A correlation technique may be used at step 618 to calculate the correlation between the base b-mode image and the current b-mode image. For example, techniques such as least squares, contour-based segmentation, or any other correlation method may be used. At step 620, the processor 116 determines if the correlation is larger than a threshold in order to determine if the position of the probe 106 has changed since the base b-mode image was acquired. Since, according to an exemplary embodiment, the emitter 122 of the needle tracking system is disposed in the probe 106, it is important that the probe remains stationary when acquiring needle position data to calculate the needle position. If the probe 106 has moved more than the threshold amount, the change in the needle position calculated at step 614 will not be accurate. It may not be possible for the processor 116 to determine if delta theta and delta phi are due to non-axial movement of the hub 128 or from movement of the probe 106. Therefore, if the base needle image and the current needle image are poorly correlated (i.e. if the correlation is less than the threshold), the method 600 advances to step 622. If the base image and the current needle image are poorly correlated, that would tend to indicate that the probe 106 has been moved. At step 622, the processor 116 empties the base needle position and the base b-mode image, and the method 600 then proceeds to step 602.

If, however, the correlation between the base b-mode image and the current b-mode image is greater than the threshold at step 620, the method 600 advances to step 624. At step 624, the processor 116 calculates that the risk of bending for the needle is significant since the correlation was above the threshold at step 620. Next, at step 626, the processor 116 presents the risk of bending. Displaying the risk of bending may include displaying a graphic on the image to represent the risk of bending, displaying a text-based warning or message, or playing an audible warning. After performing step 626, the method 600 may return to step 602 and the previously described steps may be repeated for multiple iterations. The embodiment represented by the method 600 is advantageous because the processor 116 is able to separate changes in the needle position that are cause by probe motion from changes in the needle position that are the result of the clinician moving the needle 90 in a non-axial manner. For purposes of this disclosure, the term non-axial is defined to include movements of the needle 90 or the hub 128 in a direction other than along the needle axis 522 or trajectory defined by the base needle position. Of course, in order to be considered non-axial, the movements must exceed a threshold in a non-axial direction to be considered as presenting a significant risk of bending for the needle 90.

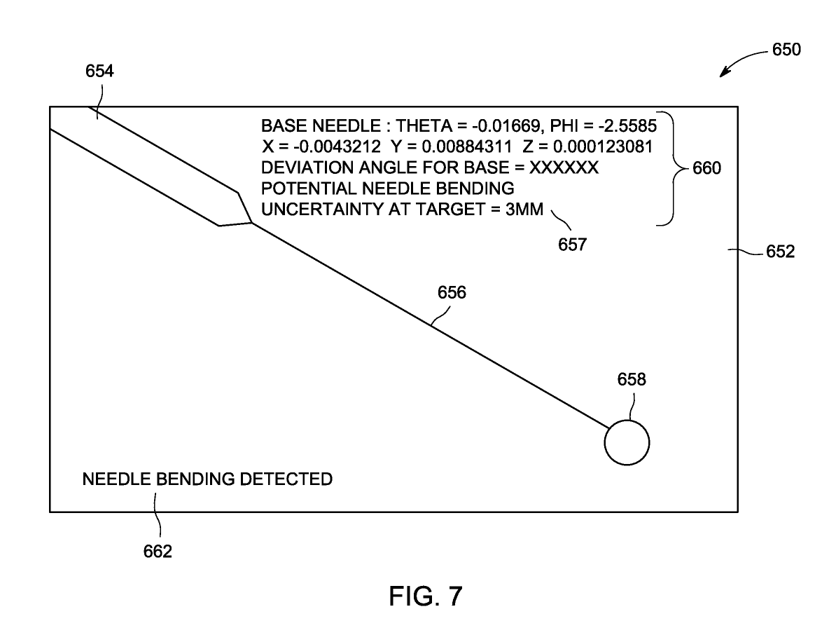

FIGS. 7, 8, 9, 10, 11, 12, and 13 are schematic representations of screenshots in accordance with various embodiments. FIGS. 7, 8, 9, 10, 11, 12, and 13 each show one or more techniques of presenting a risk of bending to a user. However, it should be appreciated that the risk of bending may be presented to the user in additional ways as well. Additionally, other embodiments may include combinations of two or more of the techniques for presenting the risk of bending shown in FIGS. 7, 8, 9, 10, 11, 12, and 13.

FIG. 7 is a schematic representation of a screenshot 650 in accordance with an embodiment. The screenshot 650 includes an ultrasound image 652, a representation of a needle 654, a projected trajectory 656, an expected target region 658, position and orientation information 660, and a text-based warning 662. The representation of the needle 654 may be based on ultrasound data, position data from a sensor, such as sensor 124 shown in FIG. 2. Or the representation of the needle 654 may be based on a combination of both ultrasound data and position data. The projected trajectory 656 is calculated based on the position data and represents a projected path in an axial direction from the representation of the needle 654. Presenting the risk of bending may include displaying the expected target region 658 for the needle. For instance, the expected target region 658 may be a circle and the diameter of the circle may be based on the risk of bending calculated by the processor 116 (shown in FIG. 1). The expected target region 658 may be any shape according to other embodiments. The size of the expected target region 658 may change based on the risk of bending. For example, the expected target region 658 may be smaller when there is relatively little risk of bending. The expected target region 658 may be larger where there is a relatively greater risk of bending.

Presenting the risk of bending may include displaying a text-based warning. For example, the text-based warning 662 includes a message indicating to a user that there is significant risk of bending. For example, the text-based warning 662 states, “needle bending detected” to alert the user that the risk of the needle bending exceeds a threshold. It should be appreciated that the specific language used in the text-based warning 662 may vary according to other embodiments. Additionally, multiple different text-based warnings may be used in order to indicate the probability of the risk of bending. Specific language may be used to differentiate a higher probability of bending from a smaller probability of bending. The position and orientation information 660 provides the user with real-time position and orientation information for the needle. Additionally the position and orientation information 660 may quantitatively indicate to the user the amount that the needle or hub has deviated from a base needle position. The position and orientation 660 may optionally include a numerical value 657 indicating the uncertainty in an expected target position for the needle due to the risk of bending.

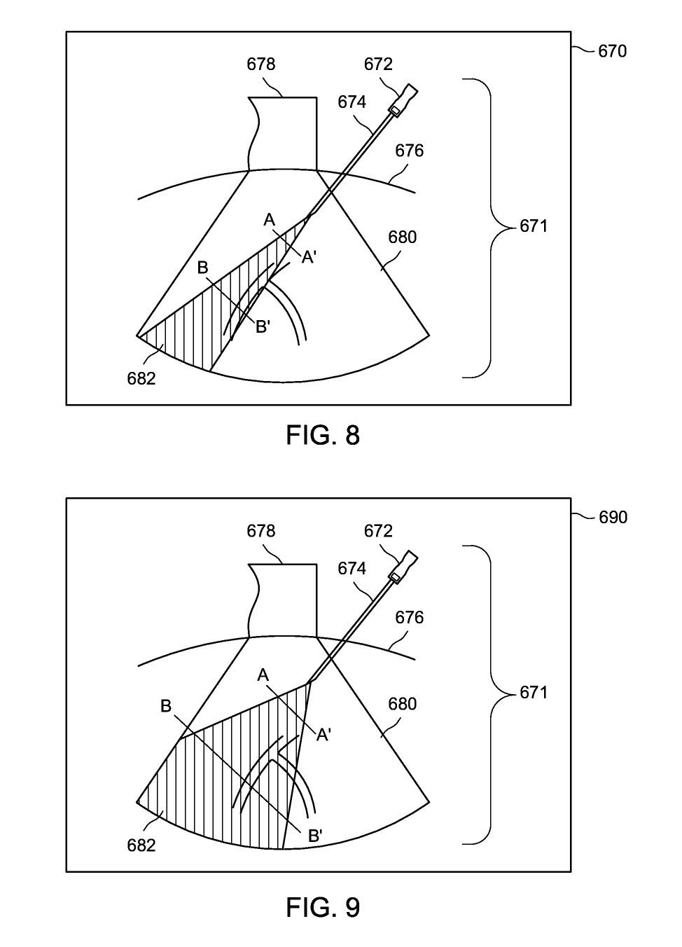

FIG. 8 is a schematic representation of a screenshot 670 in accordance with an embodiment. The screenshot 670 includes a visual representation 671. The visual representation includes a representation of a hub 672, a representation of a hollow tube 674 of the needle, a skin line 676, a representation of a probe 678, an ultrasound image 680, and an expected target region 682. According to other embodiments, the visual representation may include at least one of a representation of the probe 678 and the skin line 676. The expected target region 682 is one example of a graphic that may be displayed to present the risk of bending for the needle 90. It should be appreciated that other graphics may be used in accordance with other embodiments. The skin line 676, the representation of the hub 672, the representation of the hollow tube 674, and the representation of the probe 678 are all calculated by the processor 116 (shown in FIG. 1) based on position data. The expected target region 682 represents an area within the ultrasound image 680 where the needle is expected based on the calculated risk of bending. In accordance with an embodiment, the expected target region 682 has a width in a direction perpendicular to the direction of needle insertion that increases in a depth direction. For example, the expected target region is narrower along line A-A′ than along line B-B′. Line B-B′ is at a greater depth than line A-A′ and the extra width of the expected target region 682 at the depth of line B-B′ represents an increased uncertainty in the expected needle position due to a risk of bending.

FIG. 9 is a schematic representation of a screenshot 690 in accordance with an embodiment. The screenshot 690 includes visual representation 671. The Screenshot 690 represents a modification of the screenshot 670 (FIG. 8) that may be used to present an increased risk of bending. Common reference numbers are used in FIG. 8 and FIG. 9 to identify common elements. The expected target region 682 in FIG. 9 is wider than the expected target region 682 in FIG. 8. For example, in FIG. 9, the expected target region 682 is wider at a first depth along A-A′ and at a second depth along line B-B′ than the expected target region 682 in FIG. 8. According to an embodiment, the size and/or width of the expected target region 682 may be dynamically updated in response to changes in the risk of bending. For example, in screenshot 690, the expected target region 682 is wider than the expected target region 682 in screenshot 670 in order to present the increased risk of bending to the user. According to an embodiment, the size of the expected target region 682 may be adjusted in real-time as the processor 116 (shown in FIG. 1) updates the risk of bending of the needle through a process such as the method 400 or the method 600.

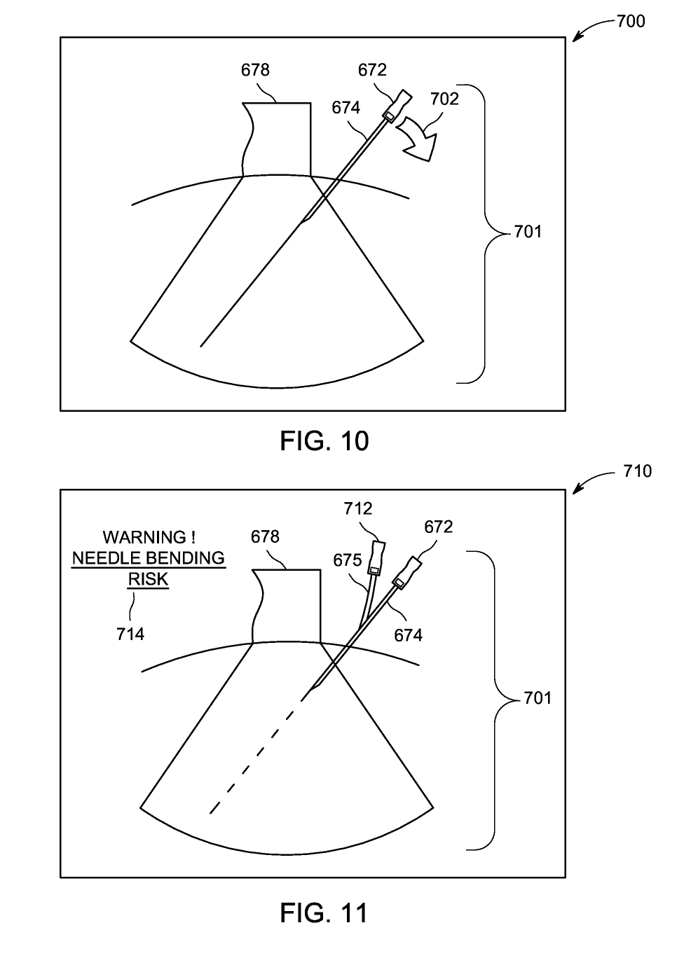

Other embodiments may include generating an icon to represent that the hub has been displaced. For example, FIG. 10 is a schematic representation of a screenshot 700 in accordance with an embodiment. The screenshot 700 includes a visual representation 701 including an arrow 702 to indicate that the needle has been displaced in a non-axial direction. The arrow 702 may be positioned with respect to the representation of the hub 672 in order to clearly indicate to the user that the hub has been displayed in direction that increases the risk of bending the needle. According to an embodiment, the arrow 702 may be positioned to indicate the direction in which the hub has been displaced so that the user may take appropriate corrective action. For example, the arrow 702 may point in different directions to indicate the non-axial displacement direction. According to other embodiments, the arrow may be rendered as a volume-rendered solid (not shown) in order to depict situations where the hub has been displaced in a direction outside of the plane of the image.

The arrow 702 shown in FIG. 10 is just one example of an icon that may be used to indicate that the hub has been displaced in a non-axial direction. It should be appreciated that other icons may be used in accordance with other embodiments. The icons may be used to simply indicate that the hub has been displaced in a non-axial direction, or the position of the icon and/or the type of icon may be used to indicate the direction of the displacement of the hub or the needle in a non-axial direction.

FIG. 11 is a schematic representation of a screenshot 710 in accordance with an embodiment. The screenshot 710 includes both the representation of the hub 672 and a second representation of the hub 712. The screenshot 710 also includes the representation of the hollow tube 674 and a second representation of the hollow tube 675. The second representation of the hollow tube 675 is shaped differently than the representation of the hollow tube 674. The second representation of the hub 712 is offset from the representation of the hub 672 in a non-axial direction. The second representation of the hub 712 and/or the second representation of the hollow tube 675 may be displayed in a different color and/or a different transparency than the representation of the hub 672. The second representation of the hub 712, and the second representation of the hollow tube 675, clearly show the user that the hub has been displaced. Additionally, a text-based warning 714 is included in the screenshot 710 to present the risk of bending. The text-based warning 714 may be replaced or supplemented with an audible warning indicating that the risk of bending has exceeded a predetermined threshold. The audible warning may be used in combination with any of the embodiments and it may comprise an alarm or a recorded message conveying the risk of needle bending. The second representation of the hub 712 and the second representation of the needle 675 graphically present the risk of bending to a user.

According to other embodiments, a representation of the needle or at least a portion of the needle may be modified to present the risk of bending to a user. For example, the representation of the hub 672 and/or the representation of the hollow tube 675 may be modified to present the risk of bending. While FIG. 11 shows both a first representation of the hub 672 and a second representation of the hub 712 to present the risk of bending, other embodiments may rely instead on simply modifying the representation of the hub 672. For example, the representation of the hub 672 may be modified through one or more of the following list of attributes: location, color, transparency, or any other graphical property of the representation of the hub 672. Likewise, the representation of the hollow tube 674 may be modified, either alone or in combination with the representation of the hub 672 to present the risk of bending. The representation of the hollow tube 674 may be modified through one or more of the following list of attributes: shape, position, color, transparency, or any other graphical property of the representation of the hollow tube 674. According to an exemplary embodiment, the representation of the hub 672 may be moved to the position of the second representation of the hub 712, and the representation of the hollow tube 674 may be modified to the shape and position of the second representation of the hollow tube 675 in order to present the risk of bending. The representation of the needle or a portion of the needle may be modified in other ways in accordance with additional embodiments to present the risk of bending.

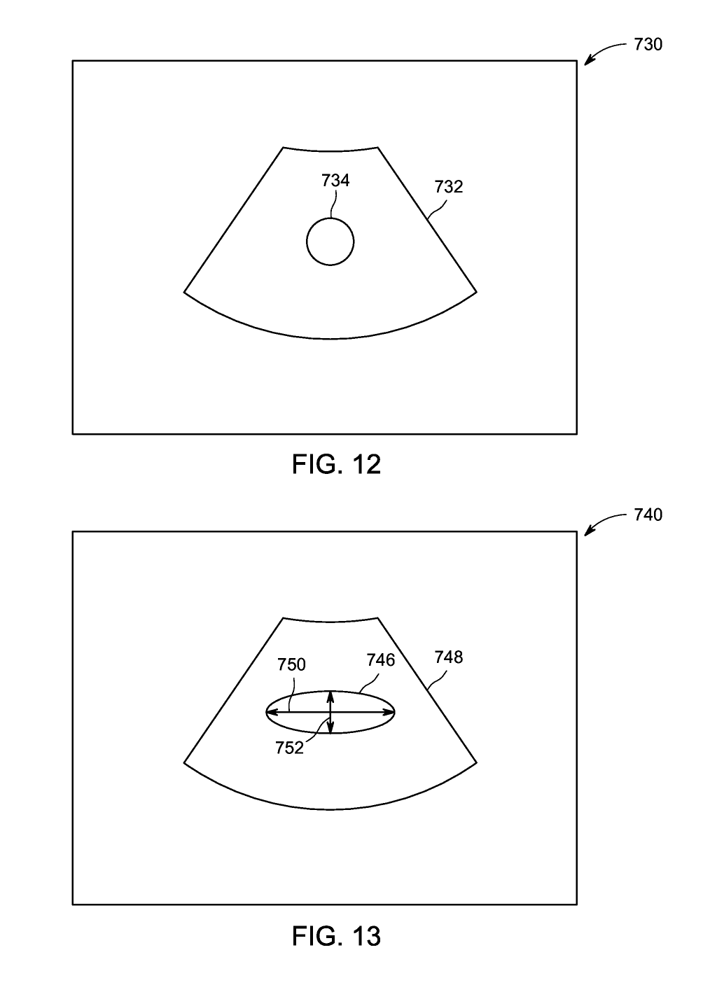

FIG. 12 is a schematic representation of a screenshot 730 in accordance with an embodiment where the needle is inserted from out-of-plane. Screenshot 730 includes an ultrasound image 732 and an expected target region 734. The expected target region 734 is shown as a circle in FIG. 12, but the expected target region 734 could be any other shape as well. The size or radius of the expected target region 734 may represent the uncertainty in an expected target position for the needle due to the risk of bending. Additionally, the size and/or dimensions of the expected target region may be updated based on the risk of bending of the needle. FIG. 12 is a schematic representation according to an embodiment where the needle is inserted from out-of-plane. Screenshot 740, shown in FIG. 13, includes an expected target region 746 and an ultrasound image 748. The expected target region 746 is oval and it is wider in a long-axis direction 750 than in a short-axis direction 752. The oval shape of the expected target region 746 indicates that there is a greater risk of bending in the long-axis direction 750 than in the short-axis direction 752. The size and orientation of the expected target region 746 may be updated in real-time to reflect the changing risk of bending of the needle. The expected target region 734 in FIG. 12 indicates that the risk of bending is the same in all directions. According to an embodiment, the size and shape of the expected target region may be adjusted as the risk of bending changes to provide real-time feedback to the user.

FIGS. 7, 8, 9, 10, 11, 12, and 13 each depict various ways of presenting the risk of bending for the needle to the user according to various embodiments. By presenting the risk of bending to the user either before or during the process of inserting the needle, the user is able to access, in real-time, whether the risk of bending is acceptable, or whether one or more corrections should be made during the process of inserting the needle to reduce the risk of bending. It should be appreciated that the Figures described above are exemplary embodiments and that the risk of bending for the needle may be presented according in other ways according to other embodiments.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. A method of ultrasound guidance for interventional procedures involving a needle, the method comprising:
acquiring ultrasound data from a region of interest;
positioning the needle with respect to the region of interest;
displaying an image based on the ultrasound data;
calculating a risk of bending for the needle; and
presenting the risk of bending for the needle.

2. The method of claim 1, wherein the risk of bending is calculated based on one or more of the following factors: a gauge of the needle, a stiffness of the needle, a depth of penetration of the needle, movement of a hub of the needle in a non-axial direction, and whether or not the needle has penetrated the skin.

3. The method of claim 1, wherein the risk of bending is calculated based on a stiffness or a gauge of the needle, a depth of penetration, and movement of a hub of the needle in a non-axial direction.

4. The method of claim 1, wherein presenting the risk of bending comprises displaying an expected target region for the needle based on the risk of bending, where a size of the expected target region represents an uncertainty in an expected target position for the needle due to the risk of bending.

5. The method of claim 1, wherein presenting the risk of bending comprises displaying a numerical value indicating an uncertainty in an expected target position for the needle due to the risk of bending.

6. The method of claim 1, further comprising displaying a representation of at least a portion of the needle, and wherein presenting the risk of bending comprises modifying the representation of at least the portion of the needle.

7. The method of claim 1, further comprising displaying a representation of the hub at the same time as the image, and wherein presenting the risk of bending comprises modifying the representation of the hub.

8. The method of claim 7, further comprising displaying a representation of a hollow tube of the needle, and wherein presenting the risk of bending further comprises modifying the representation of the hollow tube.

9. A method of ultrasound guidance for interventional procedures involving a needle, the method comprising:
acquiring ultrasound data from a region of interest;
positioning the needle with respect to the region of interest;
acquiring position data during the process of positioning the needle;
calculating a risk of bending for the needle based on the position data during the process of positioning the needle;
displaying an image based on the ultrasound data;
displaying a graphic on the image representing the risk of bending; and
modifying the graphic in response to an increase or a decrease in the risk of bending during the process of positioning the needle.

10. The method of claim 9, wherein the image is displayed as part of a visual representation including a representation of a skin line and a representation of a hub of the needle above the skin line.

11. The method of claim 10, wherein the graphic comprises an expected target region, and wherein said modifying the graphic comprises adjusting at least one of a size and a shape of the expected target region.

12. The method of claim 11, wherein the expected target region comprises a circle, and wherein said adjusting at least one of a size and a shape comprises adjusting a radius of the circle.

13. The method of claim 11, wherein the expected target region comprises an oval with a long-axis direction and a short-axis direction, where the risk of bending is greater in the long-axis direction, and wherein adjusting at least one of the size and a shape comprises adjusting a length in at least one of the long-axis direction and the short-axis direction.

14. The method of claim 11, wherein the expected target region comprises a shape with a width in a direction perpendicular to a direction of needle insertion that increases in a depth direction in order to represent an increased uncertainty in the expected needle position at greater depths due to the risk of bending.

15. The method of claim 10, wherein the visual representation is an out-of-plane representation.

16. The method of claim 10, wherein the graphic comprises an icon to represent that the hub has been displaced in a non-axial direction.

17. The method of claim 16, wherein the icon comprises an arrow positioned with respect to the representation of the hub.

18. The method of claim 17, wherein the arrow indicates a direction in which the hub has been displaced.

19. The method of claim 16, wherein the icon comprises a second representation of the hub that is offset in a non-axial direction from the representation of the hub.

20. An apparatus for providing ultrasound guidance for interventional procedures involving a needle, the apparatus comprising:
a needle tracking system that provides needle position data; and
an ultrasound imaging system including a processor, a probe, and a display device, wherein the processor is configured to:
receive needle position data from the needle tracking system:
control the ultrasound imaging system to acquire ultrasound data from a region of interest with the probe;
generate an image based on the ultrasound data;
display the image on the display device;
calculate a risk of bending for the needle; and
display a graphic on the image representing the risk of bending.

21. The apparatus of claim 20, wherein the graphic comprises the expected target region, and wherein the processor is configured to calculate the expected target region based on the position data and the risk of bending.

22. The apparatus of claim 21, wherein the processor is configured to change at least one of a size and a shape of the expected target region in real-time in response to a change in the risk of bending of the needle.

23. The apparatus of claim 20, wherein the processor is configured to determine if the probe has moved during the process of calculating the risk of bending for the needle, and wherein the processor is configured to determine that there is a risk of bending only if the probe has been moved less than a threshold amount.
